(19) United States
(12) Patent Application Publication
(10) Pub. No.: US 2023/0098285 A1
(43) Pub. Date: Mar. 30, 2023

(54) APPARATUS AND METHOD FOR GENERATING A PROTEIN-DRUG INTERACTION PREDICTION MODEL FOR PREDICTING PROTEIN-DRUG INTERACTION AND DETERMINING ITS UNCERTAINTY, AND PROTEIN-DRUG INTERACTION PREDICTION APPARATUS AND METHOD

(71) Applicant: SEOUL NATIONAL UNIVERSITY R&DB FOUNDATION, Seoul (KR)

(72) Inventors: Won Ho JHE, Seoul (KR); Q Hwan KIM, Seoul (KR)

(21) Appl. No.: 17/952,514

(22) Filed: Sep. 26, 2022

(30) Foreign Application Priority Data
Sep. 24, 2021 (KR) 10-2021-0126415
Apr. 12, 2022 (KR) 10-2022-0045226

(51) Int. Cl. G16B 15/30; G16C 20/50; G16B 5/00; G06N 3/04

(52) U.S. Cl. CPC G16B 15/30; G16C 20/50; G16B 5/00; G06N 3/0454

(57) ABSTRACT

An apparatus for generating a protein-drug interaction prediction model according to an aspect includes a data collection unit configured to collect protein data, drug molecular data, and interaction data between a protein and a drug molecule, a phenotype generation unit configured to generate protein phenotype data from the protein data, and generate drug molecular phenotype data from the drug molecular data, and a model generation unit configured to train a Bayesian neural network using the protein phenotype data, the drug molecular phenotype data, and the interaction data as training data to generate a protein-drug interaction prediction model.

### CROSS-REFERENCE TO RELATED APPLICATION AND CLAIM OF PRIORITY

This application claims the benefit under 35 USC § 119 of Korean Patent Application Nos. 10-2021-0126415, filed on Sep. 24, 2021, in the Korean Intellectual Property Office and 10-2022-0045226, filed on Apr. 12, 2022, in the Korean Intellectual Property Office, the entire disclosures of which are incorporated herein by reference for all purposes.

### BACKGROUND

### 1. Field of the Invention

The present invention relates to techniques for predicting a protein-drug interaction and determining an uncertainty of the prediction.

### 2. Description of the Related Art

Screening for potential drug candidates is an important process in determining the success or failure in the early stage of drug development. Screening is an operation for selecting candidate molecules which interact with a target protein that causes a disease. Only 1 percent or less of the whole molecular pool may be the candidate molecules. Since the experiments and computer simulations that have been used for screening so far are time consuming and expensive, new techniques capable of reducing the same are required.

Researches for applying artificial intelligence (AI) to the screening process have been in the spotlight recently. By using artificial intelligence, complex functional models for predicting a relationship between a representation (i.e., phenotype) of proteins and molecules and an interaction therebetween can be generated based on data. For computer simulation, a long simulation time is required in order to calculate an interaction free energy, but since training using artificial intelligence allows a series of complex processes to be compressed into a functional model, it has an advantage of obtaining results almost immediately when inputting data.

Meanwhile, existing techniques have focused on accurately predicting the prediction results of protein-drug interactions. However, if focusing only on the accurate predictions, overfitting, which refers to a model that was trained too much on the given data, occurs. If overfitting occurs, when inference data is far out of the range of training data, the model is more likely to lead to a wrong prediction.

Another issue overlooked by researchers in the existing techniques is the quality of the training data. If there is no noise, the model may perform ideal learning on the training data and all development capabilities can focus on increasing the accuracy of the model, but in real situations, most users have a small amount of data with noise. In this case, even if using a good model, good prediction results may not be obtained. In order to overcome this limitation, a method for evaluating the accuracy of a dataset by evaluating an uncertainty thereof is required from a user's point of view.

### SUMMARY

It is an object of the present invention to provide an apparatus and method for generating a protein-drug interaction prediction model for predicting a protein-drug interaction and determining an uncertainty thereof, and an apparatus and method for predicting a protein-drug interaction.

To achieve the above object, according to an aspect of the present invention, there is provided an apparatus for generating a protein-drug interaction prediction model, the apparatus including: a data collection unit (also referred to as a data collector) configured to collect protein data, drug molecular data, and interaction data between a protein and a drug molecule; a phenotype generation unit (also referred to as a phenotype generator) configured to generate protein phenotype data from the protein data, and generate drug molecular phenotype data from the drug molecular data; and a model generation unit (also referred to as a model generator) configured to train a Bayesian neural network using the protein phenotype data, the drug molecular phenotype data, and the interaction data as training data to generate a protein-drug interaction prediction model.

The phenotype generation unit may generate drug molecular phenotype data of a graph structure from the drug molecular data, and may generate protein phenotype data from the protein data using the protein phenotype generation model generated through transfer learning.

The Bayesian neural network may include: a one-dimensional convolutional network to which dropout is applied; a graph network to which dropout is applied; a combining layer; and a fully connected network to which dropout is applied;

The one-dimensional convolutional network may update the protein phenotype data, the graph network may update the drug molecular phenotype data, the combining layer may combine the updated protein phenotype data and the updated drug molecular phenotype data to generate combined data, and the fully connected network may receive the combined data and output a predictive value of the interaction between the protein and the drug molecule.

The protein data may be one-dimensional character string sequence data consisting of an arrangement of amino acid characters, and the drug molecular data may be simplified molecular-input line-entry system (SMILES) data in which a structure of molecules is represented as a one-dimensional character string.

In addition, according to another aspect of the present invention, there is provided an apparatus for predicting a protein-drug interaction, the apparatus including: a data acquisition unit configured to acquire protein data and drug molecular data; a phenotype generation unit configured to generate protein phenotype data from the protein data, and generate drug molecular phenotype data from the drug molecular data; and an interaction prediction unit configured to, by using a protein-drug interaction prediction model generated by training a Bayesian neural network, predict an interaction between a protein and a drug molecule based on the protein phenotype data and the drug molecular phenotype data, and determine an uncertainty of the prediction.

The phenotype generation unit may generate drug molecular phenotype data of a graph structure from the drug molecular data, and may generate protein phenotype data from the protein data using a protein phenotype generation model generated through transfer learning.

The Bayesian neural network may be a Bayesian neural network to which dropout is applied, and the interaction prediction unit may predict the interaction between the protein and the drug molecule a plurality of times by applying dropout, and may determine a final predictive value of the interaction between the protein and the drug molecule and the uncertainty of the final predictive value based on the prediction results of the plurality of times.

The interaction prediction unit may determine the final predictive value by averaging the prediction results of the plurality of times, and may determine the uncertainty of the final predictive value from a distribution of the prediction results of the plurality of times.

The uncertainty of the final predictive value may include an epistemic uncertainty and an aleatoric uncertainty.

The interaction prediction unit may determine the epistemic uncertainty using an equation below:

$$E . U . = \frac{1}{T} {\sum }_{t = 1}^{T} ( {\hat{y}}_{t}^{*} - \overline{y} ) {( {\hat{y}}_{t}^{*} - \overline{y} )}^{T} \qquad [ Equation ]$$

(wherein, E.U. represents the epistemic uncertainty, T represents the number of predictions, ŷₜ* represents the t-th prediction result, and y̅ represents an average value of the predictions).

The interaction prediction unit may determine the aleatoric uncertainty using an equation below:

$$A . U . = \frac{1}{T} {\sum }_{t = 1}^{T} [ diag ( {\hat{y}}_{t}^{*} ) - ( {\hat{y}}_{t}^{*} ) {( {\hat{y}}_{t}^{*} )}^{T} ] \qquad [ Equation ]$$

(wherein, A.U. represents the aleatoric uncertainty, T represents the number of predictions, and ŷₜ* represents the t-th prediction result).

Further, according to another aspect of the present invention, there is provided a method for predicting a protein-drug interaction, the method including: acquiring protein data and drug molecular data; generating protein phenotype data from the protein data; generating drug molecular phenotype data from the drug molecular data; and by using a protein-drug interaction prediction model generated by training a Bayesian neural network, predicting an interaction between a protein and a drug molecule based on the protein phenotype data and the drug molecular phenotype data, and determining an uncertainty of the prediction.

The step of generating the protein phenotype data may include generating protein phenotype data from the protein data using a protein phenotype generation model generated through transfer learning, and the step of generating the drug molecular phenotype data may include generating drug molecular phenotype data of a graph structure from the drug molecular data.

The Bayesian neural network may be a Bayesian neural network to which dropout is applied, and the step of predicting the interaction between the protein and the drug molecule and determining the uncertainty of the prediction may include predicting the interaction between the protein and the drug molecule a plurality of times by applying dropout, and determining a final predictive value of the interaction between the protein and the drug molecule and the uncertainty of the final predictive value based on the prediction results of the plurality of times.

The step of determining the final predictive value and the uncertainty of the final predictive value may include determining the final predictive value by averaging the prediction results of the plurality of times, and determine the uncertainty of the final predictive value from a distribution of the prediction results of the plurality of times.

The uncertainty of the final predictive value may include an epistemic uncertainty and an aleatoric uncertainty.

The step of determining the uncertainty of the final predictive value may include determining the epistemic uncertainty using an equation below:

$$E . U . = \frac{1}{T} {\sum }_{t = 1}^{T} ( {\hat{y}}_{t}^{*} - \overline{y} ) {( {\hat{y}}_{t}^{*} - \overline{y} )}^{T} \qquad [ Equation ]$$

(wherein, A.U. represents the epistemic uncertainty, T represents the number of predictions, ŷₜ* represents the t-th prediction result, and y̅ represents an average value of the predictions).

The step of determining the uncertainty of the final predictive value may determine the aleatoric uncertainty using an equation below:

$$A . U . = \frac{1}{T} {\sum }_{t = 1}^{T} [ diag ( {\hat{y}}_{t}^{*} ) - ( {\hat{y}}_{t}^{*} ) {( {\hat{y}}_{t}^{*} )}^{T} ] \qquad [ Equation ]$$

(wherein, A.U. represents the aleatoric uncertainty, T represents the number of predictions, and ŷₜ* represents the t-th prediction result).

According to the present invention, the prediction of protein-drug interaction and the uncertainty thereof are determined using a Bayesian neural network, such that the quality of data may be checked, and the reliability of the prediction may be increased by a method of removing low-quality data from the data pool.

In addition, the prediction uncertainty may be analyzed by dividing it into a data-based uncertainty (hereinafter, an “aleatoric uncertainty”) and a model-based uncertainty (hereinafter, an “epistemic uncertainty), such that, if the epistemic uncertainty is high, it is possible to further add data, and if the aleatoric uncertainty is high, it is possible to use a method of performing an experiment again or removing the data from the dataset in the actual development site.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a block diagram illustrating a protein-drug interaction prediction system according to an exemplary embodiment;

FIG. 2 is a block diagram illustrating an apparatus for generating a protein-drug interaction prediction model according to an exemplary embodiment;

FIG. 3 is a block diagram illustrating a structure of a Bayesian neural network according to an exemplary embodiment;

FIG. 4 is a block diagram illustrating a structure of a protein-drug interaction prediction apparatus according to an exemplary embodiment;

FIG. 5 is a diagram illustrating a state of exchanging a message between nodes and edge vectors of a graph network;

FIG. 6 is a block diagram illustrating and describing a computing environment including a computing device suitable for use in exemplary embodiments;

FIG. 7 is a flowchart illustrating a procedure of a method for generating a protein-drug interaction prediction model according to an exemplary embodiment;

FIG. 8 is a flowchart illustrating a procedure of a protein-drug interaction prediction method according to an exemplary embodiment;

FIG. 9 are tables illustrating prediction accuracy of datasets according to the embodiment;

FIG. 10 is a table illustrating values obtained by evaluating aleatoric uncertainty according to an embodiment; and

FIG. 11 is graphs illustrating changes in prediction accuracy according to dataset screening.

### DETAILED DESCRIPTION OF THE INVENTION

Hereinafter, embodiments will be described in detail with reference to the accompanying drawings. In denoting reference numerals to components of respective drawings, it should be noted that the same components will be denoted by the same reference numerals although they are illustrated in different drawings. Further, in description of preferred embodiments of the present invention, the publicly known functions and configurations related to the present invention, which are verified to be able to make the purport of the present invention unnecessarily obscure will not be described in detail.

Meanwhile, in respective steps, each of the steps may occur differently from the specified order unless a specific order is clearly described in the context. That is, each of the steps may be performed in the same order as the specified order, may be performed substantially simultaneously, or may be performed in the reverse order.

Further, wordings to be described below are defined in consideration of the functions in the present invention, and may differ depending on the intentions of a user or an operator or custom. Accordingly, such wordings should be defined on the basis of the contents of the overall specification.

It will be understood that, although the terms first, second, etc. may be used herein to describe various components, but these components should not be limited by these terms. These terms are used only to distinguish one component from other components. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

In addition, a division of the configuration units in the present disclosure is intended for ease of description and divided only by the main function set for each configuration unit. That is, two or more of the configuration units to be described below may be combined into a single configuration unit or formed by two or more of divisions by function into more than a single configuration unit. Further, each of the configuration units to be described below may additionally perform a part or all of the functions among functions set for other configuration units other than being responsible for the main function, and a part of the functions among the main functions set for each of the configuration units may be exclusively taken and certainly performed by other configuration units. Each of the configuration units to be described below may be implemented as hardware or software, or may be implemented as a combination of hardware and software.

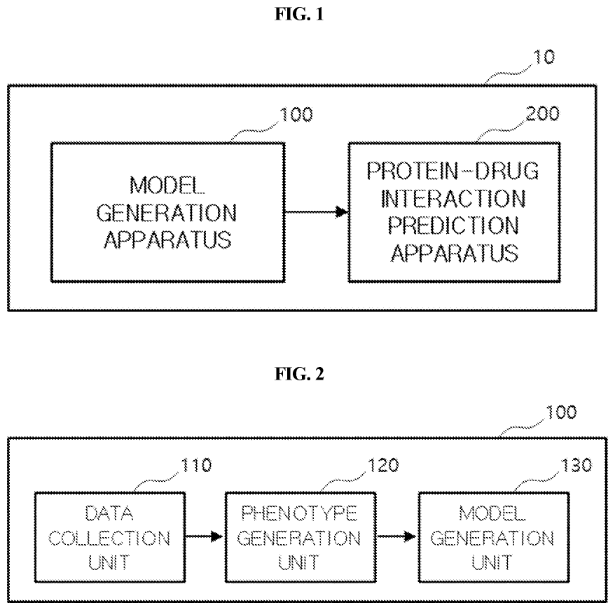

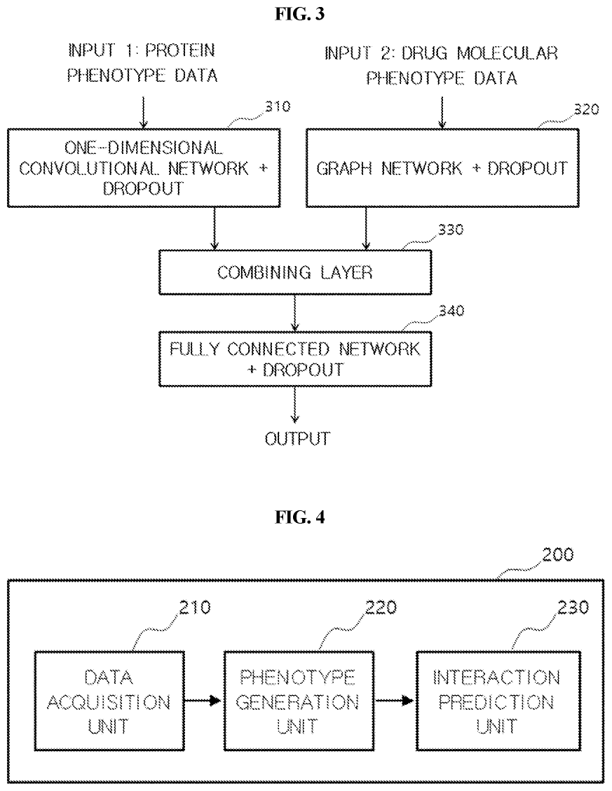

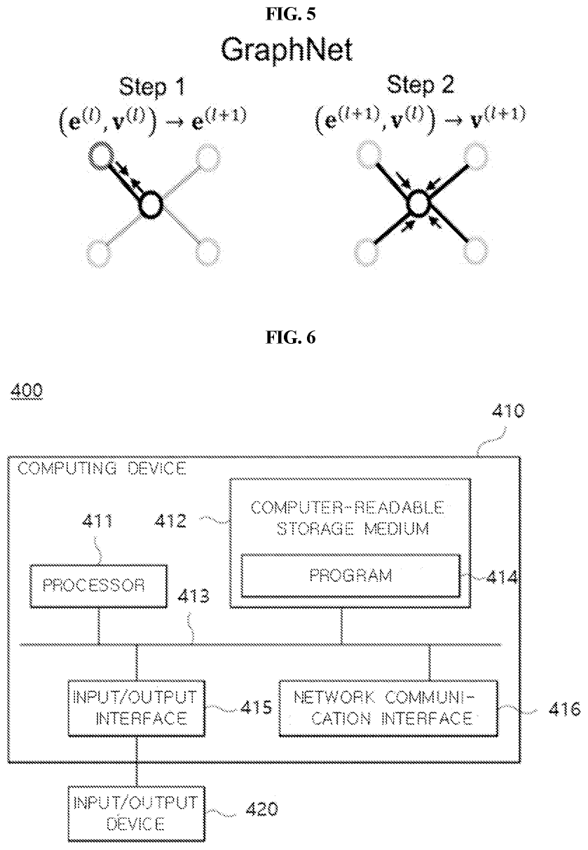

FIG. 1 is a block diagram illustrating a protein-drug interaction prediction system according to an exemplary embodiment, FIG. 2 is a block diagram illustrating an apparatus for generating a protein-drug interaction prediction model according to an exemplary embodiment, FIG. 3 is a block diagram illustrating a structure of a Bayesian neural network according to an exemplary embodiment, FIG. 4 is a block diagram illustrating a structure of a protein-drug interaction prediction apparatus according to an exemplary embodiment, and FIG. 5 is a diagram illustrating a state of exchanging a message between nodes and edge vectors of a graph network.

Referring to FIG. 1, a protein-drug interaction prediction system 10 according to an exemplary embodiment may include an apparatus for generating a protein-drug interaction prediction model (hereinafter, a ‘model generation apparatus’) 100 and an apparatus for predicting a protein-drug interaction (hereinafter, a ‘protein-drug interaction prediction apparatus) 200.

The model generation apparatus 100 may generate a protein-drug interaction prediction model capable of predicting an interaction between a protein and a drug molecule and determining an uncertainty of the prediction using a Bayesian neural network to which dropout is applied.

As shown in FIG. 2, the model generation apparatus 100 may include a data collection unit 110, a phenotype generation unit 120, and a model generation unit 130.

The data collection unit 110 may collect data on a plurality of proteins (hereinafter, protein data), data on a plurality of drug molecules (hereinafter, drug molecular data), and data on interactions between the proteins and the drug molecules (hereinafter, interaction data).

Herein, the protein data may be one-dimensional character string sequence data consisting of an arrangement of amino acid characters, and the drug molecular data may be simplified molecular-input line-entry system (SMILES) data in which a structure of molecules is represented as a one-dimensional character string.

According to an exemplary embodiment, the data collection unit 110 may collect a plurality of protein data, a plurality of drug molecular data, and interaction data between each protein and each drug molecule from an external device using a wired and/or wireless communication technique. In this case, the wireless communication technique may include Bluetooth communication, Bluetooth Low Energy (BLE) communication, Near Field Communication (NFC), wireless local area network (WLAN) communication, Zigbee communication, Infrared Data Association (IrDA) communication, Wi-Fi Direct (WFD) communication, ultra-wideband (UWB) communication, Ant+ communication, WIFI communication, Radio Frequency Identification (RFID) communication, 3G communication, 4G communication, 5G communication, or the like, but it is not limited thereto.

The phenotype generation unit 120 may generate protein phenotype data and drug molecular phenotype data to be used in generating a protein-drug interaction prediction model based on the collected protein data and the collected drug molecular data.

It is important to generate phenotypes well because they can increase the accuracy in subsequent data training and inference steps.

Proteins may have a unique three-dimensional structure through protein folding in which a one-dimensional linkage of amino acids is naturally folded inside a cell. In this case, the protein data may be represented as a one-dimensional amino acid sequence omitting the three-dimensional structure. On the other hand, the drug molecules have a much smaller size than the proteins and may consist of tens to hundreds of atoms. Therefore, the drug molecule may compress all the connection information between atoms into the one-dimensional string. SMILES may represent molecular information in a one-dimensional string in accordance with a series of rules.

According to an exemplary embodiment, since both the protein data and the drug molecular data are character strings but have different characteristics as described above, the phenotype generation unit 120 may generate protein phenotype data and drug molecular phenotype data using phenotyping techniques different from each other.

According to an exemplary embodiment, the phenotype generation unit 120 may generate protein phenotype data from the protein data using the protein phenotype generation model generated through transfer learning. By using a protein phenotype generation model generated by transfer learning a pre-training model trained with huge data, rather than one-hot encoding, which is generally used when expressing proteins, it is possible to obtain a phenotype that better reflects the characteristics of the proteins than the one-hot encoding. One-hot encoding reflects only individual information of amino acids, but when using the protein phenotype generation model generated by transfer learning the pre-training model, it is possible to reflect a deeper level of information such as an alignment pattern of the amino acids inside the protein. For example, a pre-training model using a transformer structure may be used.

The protein phenotype generation model may be generated in advance by transfer learning the pre-training model trained with huge data as described above, and may be stored in an internal or external memory of the model generation apparatus 100.

The phenotype generation unit 120 may generate drug molecular phenotype data of a graph structure from the drug molecular data. The phenotype of the graph structure may reflect the structure of molecules closer to reality than the character string, and may efficiently train the model using a graph neural network to be described below.

The model generation unit 130 may generate a protein-drug interaction prediction model using the protein phenotype data, the drug molecular phenotype data, and the interaction data between the protein and the drug molecule as training data.

Specifically, by using the protein phenotype data, the drug molecular phenotype data, and the interaction data between the protein and the drug molecule as training data, the model generation unit 130 may train the Bayesian neural network to which dropout is applied to generate the protein-drug interaction prediction model.

According to an exemplary embodiment, as shown in FIG. 3, the Bayesian neural network may include a one-dimensional convolutional network 310, a graph network 320, a combining layer 330, and a fully connected network 340.

The one-dimensional convolutional network 310 may receive the protein phenotype data and update the protein phenotype data, and the graph network 320 may update the drug molecular phenotype data which is the phenotype of the graph structure.

The combining layer 330 may generate combined data by combining the updated protein phenotype data output from the one-dimensional convolutional network 310 with the updated drug molecular phenotype data output from the graph network 320. In this case, the combined data may be vector data.

The fully connected network 340 may receive the combined data generated through the combining layer 330 and output a predictive value of the interaction between the protein and the drug molecule.

According to an exemplary embodiment, dropout may be applied to the one-dimensional convolutional network 310, the graph network 320, and the fully connected network 340, respectively, which constitute the Bayesian neural network.

FIG. 3 illustrates an example in which the Bayesian neural network includes one each of the one-dimensional convolutional network 310, the graph network 320, and the fully connected network 340, but it is not limited thereto. That is, it is possible to include two or more of the one-dimensional convolutional networks 310, the graph networks 320, and the fully connected networks 340 depending on the characteristics of data, and dropout may be applied to some or all of the one-dimensional convolutional networks 310, the graph networks 320, and the fully connected networks 340. For example, dropout may be applied to all layers except for the combining layer 330 and an output layer of the Bayesian neural network.

By using the protein-drug interaction prediction model generated by the model generation apparatus 100, the protein-drug interaction prediction apparatus 200 may predict an interaction between a target protein and a target drug molecule and determine an uncertainty of the prediction.

As shown in FIG. 4, the protein-drug interaction prediction apparatus 200 may include a data acquisition unit 210, a phenotype generation unit 220, and an interaction prediction unit 230.

The data acquisition unit 210 may acquire target protein data and target drug molecular data. Herein, the target protein data may be one-dimensional character string sequence data consisting of an arrangement of amino acid characters, and the target drug molecular data may be SMILES data in which the structure of molecules is represented as a one-dimensional character string.

For example, the data acquisition unit 210 may receive and acquire the target protein data and the target drug molecular data from a user through a predetermined input means. In this case, the input means may include a key pad, a dome switch, a mouse, a touch pad, a jog wheel, a jog switch, a hardware/software button or the like. In particular, when the touch pad forms a layer structure together with the display, it may be referred to as a touch screen.

As another example, the data acquisition unit 210 may acquire target protein data and target drug molecular data from an external device using the wired/wireless communication technique.

The phenotype generation unit 220 may generate protein phenotype data and drug molecular phenotype data based on the target protein data and the target drug molecular data.

For example, the phenotype generation unit 220 may generate the protein phenotype data from the target protein data using the protein phenotype generation model generated through transfer learning. In addition, the phenotype generation unit 220 may generate the drug molecular phenotype data of a graph structure from the target drug molecular data.

By using the protein-drug interaction prediction model generated by the model generation apparatus 100, the interaction prediction unit 230 may predict the interaction between the target protein and the target drug molecule based on the protein phenotype data and the drug molecular phenotype data, and determine the uncertainty of the prediction. In this case, the uncertainty may include an epistemic uncertainty and an aleatoric uncertainty.

When a sequence length of the target protein is set to be Lₚ, the protein phenotype generation model may receive the target protein data and output a matrix Xₚ∈ [symbol] Lₚ×d. In this case, dimensions d of the matrix may vary depending on the size of the protein phenotype generation model. Where Xₚ represents amino acid-level data, and in order to obtain protein-level data, the matrix should be compressed into a vector. Herein, a protein vector xₚ⁽⁰⁾∈ [symbol] ^{d} may be obtained by a method of averaging Lₚ amino acid expression vectors. The protein vector has a predetermined pattern that can distinguish different proteins, and in order to effectively extract these patterns, by passing them through a one-dimensional convolutional network (310 in FIG. 3), a protein expression vector xₚ may be finally obtained.

In the drug molecular data converted into a graph structure, characteristic vectors are assigned to atoms and a bond, respectively. The atom and the bond may be expressed as a node and an edge of a graph, respectively, and may be written as vᵢ⁽⁰⁾ and eᵢⱼ⁽⁰⁾, respectively. In this case, the subscripts i and mean j th atom and a bond connecting the i th atom with the j th atom, respectively. In the nodes and an edge vector, representation is trained by exchanging messages along the connection therebetween.

According to an exemplary embodiment, the graph network (320 in FIG. 3) configured to sequentially update both the nodes and the edge may be used. As shown in FIG. 5, the graph network (320 in FIG. 3) consists of two steps of updating the edge and e) the nodes. First, an edge vector eᵢⱼ^{(l)} is updated by referring to information of two nodes on both ends of the edge. This may be expressed by Equation 1 below.

eᵢⱼ^{(l+1)}=ReLU[(eᵢⱼ^{(l)}⊕vᵢ^{(l)}⊕vⱼ^{(l)})Wₑ^{(l)}+bₑ^{(l)}] [Equation 1]

Wherein, ⊕ is a concatenation operator, Wₑ^{(l)} is a weight matrix, bₑ^{(l)} is a bias vector, and ReLU is a rectified linear unit function.

Next, a node vector vᵢ^{(l)} is updated using the updated vectors eᵢⱼ^{(l+1)}. This may be expressed by Equation 2 below.

$${v}_{i}^{( l + 1 )} = ReLU [ ( {v}_{i}^{( l )} ⊕ {\sum }_{j ∈ N ( i )} {e}_{ij}^{( l + 1 )} ) {W}_{v}^{( l )} + {b}_{v}^{( l )} ] \qquad [ Equation 2 ]$$

Wherein, Wᵥ^{(l)} and bᵥ^{(l)} are the weight matrix and the bias vector, respectively.

Atoms and the concatenated vectors that have passed through the graph network (320 of FIG. 3) should be finally expressed as one vector corresponding to one molecule. A drug molecular expression vector x_{d} may be expressed by Equation 3 below by averaging all the atoms and concatenated vectors constituting the molecule.

$${x}_{d} = ( \frac{1}{{N}_{v}} {\sum }_{i} {v}_{i} ) ⊕ ( \frac{1}{{N}_{e}} {\sum }_{i , j} {e}_{ij} ) \qquad [ Equation 3 ]$$

Wherein, Nᵥ and Nₑ are the number of nodes and edges of the graph, that is, the number of the atoms and the concatenated vectors in the molecule, respectively.

The protein expression vector xₚ and the drug molecular expression vector x_{d} are expressed as one expression vector while passing through the combining layer (330 in FIG. 3). This may be expressed by Equation 4 below.

x=xₚ⊕x_{d} [Equation 4]

The expression vector x passes through the fully connected network (340 of FIG. 3), then may be finally output as a predictive value.

According to an exemplary embodiment, the interaction prediction unit 230 may perform independent prediction on the protein phenotype data of the target protein and the drug molecular phenotype data of the target drug molecule a plurality of times using dropout, and average these predictive values to determine a final predictive value of the interaction between the target protein and the target drug molecule, and determine the uncertainty of the final predictive value from a distribution of the predictive values.

The protein phenotype data of the target protein and the drug molecular phenotype data of the target drug molecule may pass through the protein-drug interaction prediction model several times, for example T times, instead of once. If passing the same data through the protein-drug interaction prediction model to which dropout is applied T times, the input data passes through networks of different structures each time because dropout is applied thereto. Then, Monte-Carlo inference using approximately T network ensembles is possible, and an average of these T predictive values may be the final predictive value.

For example, the interaction prediction unit 230 may determine the uncertainty by dividing it into an epistemic uncertainty and an aleatoric uncertainty from the heteroscedasticity assumption that the uncertainty of the prediction may be different for each data. For example, the interaction prediction unit 230 may determine an epistemic uncertainty E.U. and an aleatoric uncertainty A.U. using Equations 5 and 6 below.

$$E . U . = \frac{1}{T} {\sum }_{t = 1}^{T} ( {\hat{y}}_{t}^{*} - \overline{y} ) {( {\hat{y}}_{t}^{*} - \overline{y} )}^{T} \qquad [ Equation 5 ]$$

$$A . U . = \frac{1}{T} {\sum }_{t = 1}^{T} [ diag ( {\hat{y}}_{t}^{*} ) - ( {\hat{y}}_{t}^{*} ) {( {\hat{y}}_{t}^{*} )}^{T} ] \qquad [ Equation 6 ]$$

Wherein, T may mean the number of predictions, ŷₜ* may mean the t-th prediction result, and y̅ may mean an average value of the predictions.

FIG. 6 is a block diagram illustrating and describing a computing environment including a computing device suitable for use in exemplary embodiments. In the illustrated embodiment, each component may have different functions and capabilities other than those described below, and the computing environment may also include additional components in addition to those described below.

A computing environment 400 illustrated in FIG. 6 may include a computing device 410. According to an embodiment, the computing device 410 may include, for example, one or more components included in the model generation apparatus 100 and/or the protein-drug interaction prediction apparatus 200, which are described with reference to FIGS. 1 to 5.

The computing device 410 may include at least one processor 411, a computer-readable storage medium 412, and a communication bus 413. The processor 411 may cause the computing device 410 to operate according to the above-described exemplary embodiments. For example, the processor 411 may execute one or more programs 414 stored in the computer-readable storage medium 412. The one or more programs 414 may include one or more computer-executable instructions. When executed by the processor 411, the computer-executable instructions may be configured to cause the computing device 410 to perform operations according to the exemplary embodiments.

The computer-readable storage medium 412 may store computer-executable instructions or program code, program data, and/or other suitable type of information. The program 414 stored in the computer-readable storage medium 412 may include a set of instructions executable by the processor 411. According to one embodiment, the computer-readable storage medium 412 may be a memory (a volatile memory, such as a random access memory, a non-volatile memory, or a suitable combination thereof), one or more magnetic disk storage devices, optical disk storage devices, flash memory, other type of storage medium accessed by the computing device 410 and capable of storing desired information, or a suitable combination thereof.

The communication bus 413 may connect various other components of the computing device 410 including the processor 411 and the computer-readable storage medium 412 with each other.

The computing device 410 may also include one or more input/output interfaces 415 and one or more network communication interfaces 416, which provide interfaces for one or more input/output devices 420. The input/output interface 415 and the network communication interface 416 may be connected to the communication bus 413. The input/output device 420 may be connected to other components of the computing device 410 through the input/output interface 415. The input/output device 420 may include, for example, a pointing device (such as a mouse or trackpad), a keyboard, a touch input device (such as a touchpad or a touchscreen), a voice or sound input device, various types of sensor devices, and/or input devices such as a photographing device, and/or output devices such as a display device, printer, speakers and/or network card. The input/output device 420 may be included in the computing device 410 as one component constituting the computing device 410 or may be connected to the computing device 410 as a separate device distinct from the computing device 410.

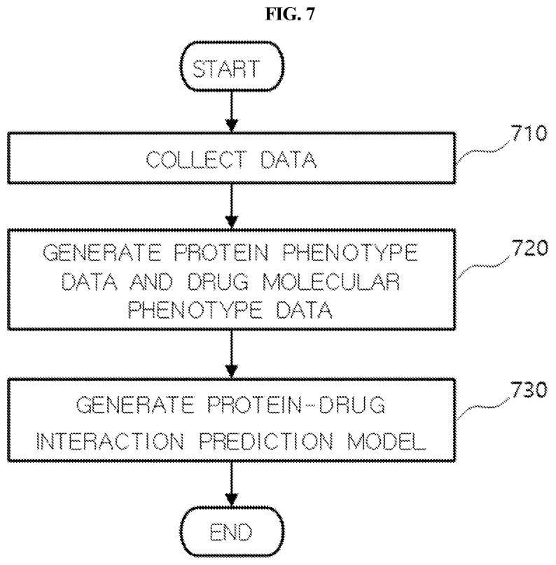

FIG. 7 is a flowchart illustrating a procedure of a method for generating a protein-drug interaction prediction model according to an exemplary embodiment. The method for generating a protein-drug interaction prediction model shown in FIG. 7 may be performed by the model generation apparatus 100 shown in FIG. 2.

Referring to FIG. 7, the model generation apparatus may collect a plurality of protein data, a plurality of drug molecular data, and interaction data between the protein and the drug molecule (710).

For example, the model generation apparatus may collect the plurality of protein data, the plurality of drug molecular data, and interaction data between each protein and each drug molecule from an external device using the wired/wireless communication technique.

Then, the model generation apparatus may generate protein phenotype data and drug molecular phenotype data to be used in generating the protein-drug interaction prediction model based on the collected protein data and the collected drug molecular data (720).

For example, the model generation apparatus may generate the protein phenotype data from the protein data using the protein phenotype generation model generated through transfer learning. In addition, the model generation apparatus may generate the drug molecular phenotype data of the graph structure from the drug molecular data.

Next, the model generation apparatus may generate a protein-drug interaction prediction model using the protein phenotype data, the drug molecular phenotype data, and the interaction data between the protein and the drug molecule as training data (730).

For example, by using the protein phenotype data, the drug molecular phenotype data, and the interaction data between the protein and the drug molecule as training data, the model generation apparatus may train the Bayesian neural network to which dropout is applied to generate the protein-drug interaction prediction model. In this case, as shown in FIG. 3, the Bayesian neural network includes the one-dimensional convolutional network 310, the graph network 320, the combining layer 330, and the fully connected network 340, and dropout may be applied to the one-dimensional convolutional network 310, the graph network 320, and the fully connected network 340.

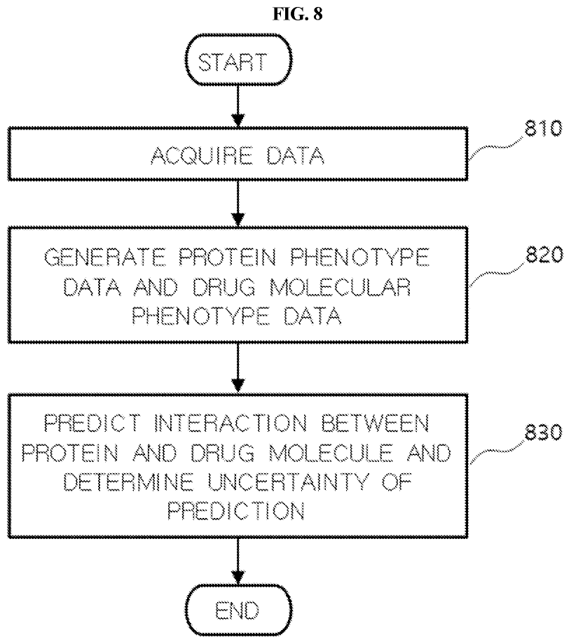

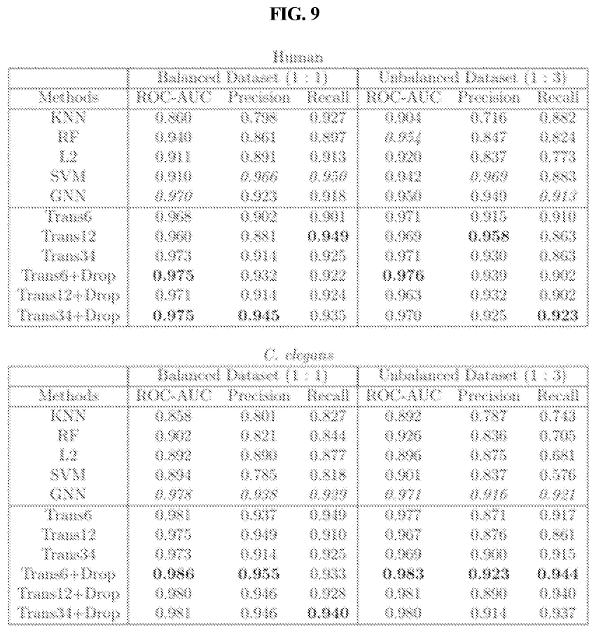

FIG. 8 is a flowchart illustrating a procedure of a protein-drug interaction prediction method according to an exemplary embodiment. The protein-drug interaction prediction method shown in FIG. 8 may be performed by the protein-drug interaction prediction apparatus 200 shown in FIG. 4.

Referring to FIG. 8, the protein-drug interaction prediction apparatus may acquire target protein data and target drug molecular data (810). Herein, the target protein data may be one-dimensional character string sequence data consisting of an arrangement of amino acid characters, and the target drug molecular data may be SMILES data in which the structure of molecules is represented as a one-dimensional character string.

For example, the protein-drug interaction prediction apparatus may receive and acquire the target protein data and the target drug molecular data from a user through a predetermined input means, or may acquire target protein data and target drug molecular data from an external device using the wired/wireless communication technique.

Then, the protein-drug interaction prediction apparatus may generate protein phenotype data and drug molecular phenotype data based on the target protein data and the target drug molecular data (820).

For example, the protein-drug interaction prediction apparatus may generate the protein phenotype data from the target protein data using the protein phenotype generation model generated through transfer learning. In addition, the protein-drug interaction prediction apparatus may generate the drug molecular phenotype data of a graph structure from the target drug molecular data.

Next, by using the protein-drug interaction prediction model, the protein-drug interaction prediction apparatus may predict the interaction between the target protein and the target drug molecule based on the protein phenotype data and the drug molecular phenotype data, and determine the uncertainty of the prediction (830). In this case, the uncertainty may include the epistemic uncertainty and the aleatoric uncertainty.

For example, the protein-drug interaction prediction apparatus may perform independent prediction on the protein phenotype data of the target protein and the drug molecular phenotype data of the target drug molecule a plurality of times using dropout, and average these predictive values to determine a final predictive value of the interaction between the target protein and the target drug molecule, and determine uncertainty of the final predictive value from the distribution of the predictive values. For example, the protein-drug interaction prediction apparatus may determine the epistemic uncertainty and the aleatoric uncertainty using Equations 5 and 6.

### Experimental Example 1—Evaluation of Prediction Accuracy Performance

Human and C. elegans datasets, which are public protein-drug interaction datasets, were used in an experiment. Two datasets had a balanced dataset and an unbalanced dataset in a ratio of positive to negative of 1:1 and 1:3, respectively. That is, a total of four datasets were used in this experiment. The Human and C. elegans datasets have 3369 and 4000 positive data, and the number of negative data is adjusted according to the ratio of the positive to negative.

As a conventional technique, five algorithms including k-nearest neighbor (KNN), random forest (RF), L2-logistic (L2), support vector machine (SVM), and graph neural network (GNN) models were used, respectively.

A total of six models were proposed by changing parameters of the structure presented in the present invention. These models were achieved by combining three types of transfer learning models (Trans6, Trans12, and Trans34) with two cases (+Drop) of applying dropout or not. The meaning of the number written after Trans indicates how many layers of the transformer structure were used by the transfer learning models. A model with +Drop indicates a Bayesian Neural Network formed by applying dropout, and a model without +Drop indicates a model to which dropout is not applied. That is, the model with +Drop indicates the technique presented in the present invention.

Results of predicting the datasets for each model are shown in FIG. 9.

FIG. 9 shows the prediction accuracy of the datasets. Three ROC-AUC, Precision, and Recall were used as a measure. In general, it can be seen that the artificial intelligence structure (Trans+Drop model) presented by the present invention exhibits the highest accuracy.

### Experimental Example 2—Evaluation of Aleatoric Uncertainty

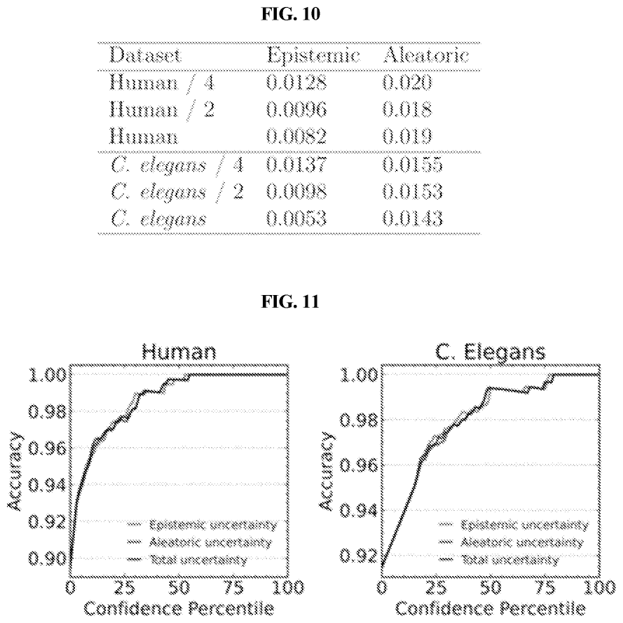

By using the public protein-drug interaction datasets, Human and C. elegans datasets, a protein-drug interaction prediction model was generated and the aleatoric uncertainty of the generated protein-drug interaction prediction model was evaluated. Evaluation results are shown in FIG. 10.

FIG. 10 is a table illustrating values obtained by evaluating the aleatoric uncertainty. In order to confirm whether the aleatoric uncertainty was correctly evaluated, a dataset reduction method was used. The uncertainty was evaluated by reducing the size of the dataset by half (/2) and quarter (/4). Since the number of training data is decreased, the epistemic uncertainty should be increased, and the aleatoric uncertainty should be constant. In this regard, FIG. 10 shows such the results well. It can be confirmed that the uncertainty of the dataset was correctly evaluated by the present invention.

When correctly evaluating the uncertainty of data, it is possible to perform an additional operation using the same. In this experiment, dataset screening of excluding dangerous data from the dataset using the uncertainty was performed. It is expected that, if the uncertainty of the data inside the dataset is measured and then removed in order of large size of the uncertainty, it will be possible to make a more accurate prediction while reducing the size of the entire dataset.

FIG. 11 is graphs illustrating changes in prediction accuracy according to dataset screening. In the graphs, confidence percentile indicates what percentage of data with large uncertainty is excluded from the dataset. In fact, it can be confirmed that the prediction accuracy is improved when data with large uncertainty is deleted.

The above-described embodiments of the present invention may be implemented as a computer-readable code in a computer-readable recording medium. The computer-readable recording medium may include all types of recording devices for storing data that may be read by a computer system. Examples of computer-readable recording medium may include ROM, RAM, CD-ROM, magnetic tape, floppy disk, optical disk and the like. Further, the computer-readable recording medium may be distributed over a computer system connected by a network, and written and implemented in computer-readable code that may be read by the computer in a distributed manner.

The present invention has been described with reference to the preferred embodiments above, and it will be understood by those skilled in the art that various modifications may be made within the scope without departing from essential characteristics of the present invention. Accordingly, it should be interpreted that the scope of the present invention is not limited to the above-described embodiments, and other various embodiments within the scope equivalent to those described in the claims are included within the present invention.

What is claimed is:

1. An apparatus for generating a protein-drug interaction prediction model, the apparatus comprising:
a data collection unit configured to collect protein data, drug molecular data, and interaction data between a protein and a drug molecule;
a phenotype generation unit configured to generate protein phenotype data from the protein data, and generate drug molecular phenotype data from the drug molecular data; and
a model generation unit configured to train a Bayesian neural network using the protein phenotype data, the drug molecular phenotype data, and the interaction data as training data to generate a protein-drug interaction prediction model.

2. The apparatus according to claim 1, wherein the phenotype generation unit is configured to generate drug molecular phenotype data of a graph structure from the drug molecular data, and generate protein phenotype data from the protein data using the protein phenotype generation model generated through transfer learning.

3. The apparatus according to claim 1, wherein the Bayesian neural network comprises:
a one-dimensional convolutional network to which dropout is applied;
a graph network to which dropout is applied;
a combining layer; and
a fully connected network to which dropout is applied.

4. The apparatus according to claim 3, wherein the one-dimensional convolutional network is configured to update the protein phenotype data;
the graph network is configured to update the drug molecular phenotype data,
the combining layer combines the updated protein phenotype data and the updated drug molecular phenotype data to generate combined data, and
the fully connected network is configured to receive the combined data and outputs a predictive value of the interaction between the protein and the drug molecule.

5. The apparatus according to claim 1, wherein the protein data is one-dimensional character string sequence data comprised of an arrangement of amino acid characters, and the drug molecular data is simplified molecular-input line-entry system (SMILES) data in which a structure of molecules is represented as a one-dimensional character string.

6. An apparatus for predicting a protein-drug interaction, the apparatus comprising:
a data acquisition unit configured to acquire protein data and drug molecular data;
a phenotype generation unit configured to generate protein phenotype data from the protein data, and generate drug molecular phenotype data from the drug molecular data; and
an interaction prediction unit configured to, by using a protein-drug interaction prediction model generated by training a Bayesian neural network, predict an interaction between a protein and a drug molecule based on the protein phenotype data and the drug molecular phenotype data, and determine an uncertainty of the prediction.

7. The apparatus according to claim 6, wherein the phenotype generation unit is configured to generate drug molecular phenotype data of a graph structure from the drug molecular data, and generate protein phenotype data from the protein data using a protein phenotype generation model generated through transfer learning.

8. The apparatus according to claim 6, wherein the Bayesian neural network is a Bayesian neural network to which dropout is applied and
the interaction prediction unit is configured to predict the interaction between the protein and the drug molecule a plurality of times by applying dropout, and determine a final predictive value of the interaction between the protein and the drug molecule and the uncertainty of the final predictive value based on the prediction results of the plurality of times.

9. The apparatus according to claim 8, wherein the interaction prediction unit is configured to determine the final predictive value by averaging the prediction results of the plurality of times, and determine the uncertainty of the final predictive value from a distribution of the prediction results of the plurality of times.

10. The apparatus according to claim 9, wherein the uncertainty of the final predictive value comprises an epistemic uncertainty and an aleatoric uncertainty.

11. The apparatus according to claim 10, wherein the interaction prediction unit is configured to determine the epistemic uncertainty using an equation:
$$E . U . = \frac{1}{T} {\sum }_{t = 1}^{T} ( {\hat{y}}_{t}^{*} - \overline{y} ) {( {\hat{y}}_{t}^{*} - \overline{y} )}^{T} \qquad [ Equation ]$$
wherein E.U. represents the epistemic uncertainty, T represents the number of predictions, ŷₜ* represents the t-th prediction result, and y̅ represents an average value of the predictions.

12. The method for claim 10, wherein the interaction prediction unit is configured to determine the aleatoric uncertainty using an equation:
$$A . U . = \frac{1}{T} {\sum }_{t = 1}^{T} [ diag ( {\hat{y}}_{t}^{*} ) - ( {\hat{y}}_{t}^{*} ) {( {\hat{y}}_{t}^{*} )}^{T} ] \qquad [ Equation ]$$
wherein A.U. represents the aleatoric uncertainty, T represents the number of predictions, and ŷₜ* represents the t-th prediction result.

13. A method for predicting a protein-drug interaction, the method comprising:
acquiring protein data and drug molecular data;
generating protein phenotype data from the protein data;
generating drug molecular phenotype data from the drug molecular data; and
by using a protein-drug interaction prediction model generated by training a Bayesian neural network, predicting an interaction between a protein and a drug molecule based on the protein phenotype data and the drug molecular phenotype data, and determining an uncertainty of the prediction.

14. The method according to claim 13, wherein the generating of the protein phenotype data comprises generating protein phenotype data from the protein data using a protein phenotype generation model generated through transfer learning; and
the generating of the drug molecular phenotype data comprises generating drug molecular phenotype data of a graph structure from the drug molecular data.

15. The method according to claim 13, wherein the Bayesian neural network is a Bayesian neural network to which dropout is applied; and
the predicting of the interaction between the protein and the drug molecule and the determining of the uncertainty of the prediction comprises predicting the interaction between the protein and the drug molecule a plurality of times by applying dropout, and determining a final predictive value of the interaction between the protein and the drug molecule and the uncertainty of the final predictive value based on the prediction results of the plurality of times.

16. The method according to claim 15, wherein the determining of the final predictive value and the uncertainty of the final predictive value comprises determining the final predictive value by averaging the prediction results of the plurality of times, and determines the uncertainty of the final predictive value from a distribution of the prediction results of the plurality of times.

17. The method according to claim 16, wherein the uncertainty of the final predictive value comprises an epistemic uncertainty and an aleatoric uncertainty.

18. The method according to claim 17, wherein the step of determining the uncertainty of the final predictive value comprises determining the epistemic uncertainty using an equation:
$$E . U . = \frac{1}{T} {\sum }_{t = 1}^{T} ( {\hat{y}}_{t}^{*} - \overline{y} ) {( {\hat{y}}_{t}^{*} - \overline{y} )}^{T} \qquad [ Equation ]$$
wherein E.U. represents the epistemic uncertainty, represents the number of predictions, ŷₜ* represents the t-th prediction result, and y̅ represents an average value of the predictions.

19. The method according to claim 17, wherein the determining of the uncertainty of the final predictive value comprising determining the aleatoric uncertainty using an equation:
$$A . U . = \frac{1}{T} {\sum }_{t = 1}^{T} [ diag ( {\hat{y}}_{t}^{*} ) - ( {\hat{y}}_{t}^{*} ) {( {\hat{y}}_{t}^{*} )}^{T} ] \qquad [ Equation ]$$
wherein A.U. represents the aleatoric uncertainty, T represents the number of predictions, and ŷₜ* represents the t-th prediction result.
